(19) United States
(12) Patent Application Publication
(10) Pub. No.: US 2012/0121671 A1
(43) Pub. Date: May 17, 2012

(54) CONTROL OF FLUID MIGRATION IN NON-LATEX BONDED WET-WIPES

(75) Inventor: Joel Erwin GOLDSTEIN, East Brunswick, NJ (US)

(73) Assignee: HERCULES INCORPORATED, Wilmington, DE (US)

(21) Appl. No.: 13/294,270

(22) Filed: Nov. 11, 2011

Related U.S. Application Data
(60) Provisional application No. 61/413,595, filed on Nov. 15, 2010.

(51) Int. Cl. A01N 25/34; A47L 13/17; B05D 1/02; A61Q 19/00; B05D 1/28; A01P 1/00; A01P 17/00; A61K 8/02; A47K 7/00; B05D 1/18

(52) U.S. Cl. 424/401; 15/104.93; 427/427.4; 427/430.1; 427/428.01; 424/403; 424/402

(57) ABSTRACT

The present invention describes wet-wipe composition with a liquid portion that is impregnated in a substrate, wherein the liquid portion contains a thickening or gelling polymer along with a thickening or gelling agent which substantially reduces fluid migration of the liquid portion within a wet-wipes stack during storage wherein the thickening or gelling agent is incorporated into the substrate through means of physical entanglement or thermal bonding.

### FIELD OF INVENTION

This application claims the benefit of U.S. Provisional Application No. 61/413,595, filed Nov. 15, 2010.

### BACKGROUND OF THE INVENTION

Wet-wipes are becoming more popular and are now being used in everyday usages as a common practice. Wet-wipes are used for cleansing the skin of adults as well as children, and especially babies because of the efficiency and gentleness of the liquid or semi liquid portion of the wipe.

Fluid migration in wet-wipes is a potential source of nuisance to a consumer. The migration results in topmost wipes in a stack becoming too dry while the bottom wipes becoming soggy and oversaturated.

The wet-wipes primarily include a substrate and a liquid portion containing benefiting agent for the particular task. For example, hard surface cleaning wipes would contain liquid with benefiting agent that would remove undesirable foreign material from the surface. On the other hand, the personal care wet-wipes would primarily include a substrate and liquid portion with a benefiting agent that would remove undesirable material from the skin or hair but may optionally leave the skin or hair feeling softer, soother, and/or healed.

Wet-wipe compositions are well documented in the prior art. Examples of this prior art is US 2005/0008680 A1 publication, incorporated herein by reference in its entirety, that discloses a wet-wipe impregnated with a lotion composition where the lotion includes a preservative system and soothing ingredients to mitigate stinging. US 2005/0009431 A1 publication, incorporated herein by reference in its entirety, discloses a process for making a wet-wipe that contains a concentrated emulsion composition of an emollient, surfactant and water wherein the surfactant solution contains at least about 25% w/w. US 2005/0266055 A1, incorporated herein by reference in its entirety, discloses a cosmetic or dermatological wipe of a water-insoluble nonwoven that is moistened with a cosmetic or dermatological composition of a Pickering water-in-oil emulsion.

One of the major issues with wet-wipes, such as baby-wipes, is migration of fluid in its container during storage. In applications such as baby-wipes, the container commonly used for storage of the wet-wipes is a top opening “tub”.

In the manufacture of wet-wipes for use as baby-wipes, the fluid is typically applied uniformly to all wipes during the manufacturing. However, fluid migrates to the bottom of the “tub” over time during storage causing the bottom towels to become very wet (soaked) with liquid fluid while causing the top layer of towels to be relatively dry. This is a major quality issue for the users and the manufacturer. Hence, there is a need in the industry to have a wet-wipe composition that has reduced or no fluid migration from the wet-wipe so that it has a longer shelf-life and improved perceived quality.

In U.S. Pat. No. 4,775,582, the fluid migration within wet-wipe stacks was to be controlled and the liquid concentration within these stacks was to be maintained though the use of particular wettable polyolefin meltblown webs.

In U.S. patent application Ser. No. 12/231,588, incorporated herein by reference in its entirety, in order to control the fluid migration during the storage of wet-wipe, a thickening or gelling polymer and the thickening or gelling agent comprise a binary system, wherein the thickening or gelling polymer in solution, dispersion or emulsion with the liquid portion comprising a continuous phase, is combined with an effective amount of the thickening or gelling agent to effectively thicken or gel the continuous phase while the continuous phase is in contact with the substrate.

Alternatively in U.S. patent application Ser. No. 12/231,588, one component of the binary system, such as a calcium containing salt may be added to a latex emulsion used in the formation of the substrate. It is a common method of web bonding by using chemical agents, which may include adhesive resins and solvents. Latex resins (adhesive) are applied to the web by a variety of methods: dipping the web into the latex and removing the excess, spraying, foaming or printing bonding. The resin is usually in a water-based solution, so this bonding process requires heat to remove the water to dry and set the binder into the fabric. This is sometimes referred to as “latex bonding”. Many types of webs use latex in their production and therefore there is an opportunity to incorporate the calcium containing salt of the binary system into the latex.

Various nonwoven substrates used in the production of wet-wipes use methods other than latex bonding in their formation, such as spunlaced, hydroentangled, mechanically entangled, needle-punched, thermally bonded or some other means. There is a need to incorporate a binary system, or a portion of a binary system into the production of these nonwoven substrate by using existing process steps as a means to incorporate an effective amount of the thickening or gelling agent into the nonwoven substrate to effectively thicken or gel the continuous phase while the continuous phase is in contact with the substrate.

### SUMMARY OF THE INVENTION

The present invention is directed to a wet-wipe having improved fluid migration properties. The wet-wipe comprises a substrate of woven, non-woven or knitted fabric in which a thickening or gelling agent, capable of thickening or gelling a thickening or gelling polymer, is incorporated into the substrate during its formation by a means other than latex bonding. The methods used to produce these substrates include spunlacing, hydroentangling, mechanically entangling, needle-punching, and thermally bonding.

Alternatively, the thickening or gelling agent may be incorporated into the continuous phase and the thickening or gelling polymer is incorporated into the substrate during its formation by a means other than latex bonding.

The wet-wipe having improved fluid migration properties also includes an aqueous phase in which contains an effective amount of a thickening or gelling polymer, which is capable of thickening or gelling the aqueous phase upon contact with the thickening or gelling agent previously incorporated into the substrate.

As used herein, the term “wet-wipe” refers to a substrate of woven, non-woven or knitted fabric which, during its manufacture, has a liquid portion applied thereto so that the liquid portion can be retained on or within the substrate until its utilization by a consumer. The liquid portion may include a fragrance and/or an emollient and may serve to aid the substrate in retention of materials which are to be wiped up during its utilization.

The thickening or gelling polymer and the thickening or gelling agent comprise a binary system, wherein the thickening or gelling polymer in solution, dispersion or emulsion with the liquid portion comprising a continuous phase, is combined with an effective amount of the thickening or gelling agent previously incorporated into the substrate to effectively thicken or gel the continuous phase while the continuous phase is in contact with the substrate.

The liquid portion can be any solution which can be absorbed into the wet-wipe fibrous sheet and may include any suitable components which provide the desired wiping properties.

### DETAILED DESCRIPTION OF THE INVENTION

Each individual wet-wipe, comprising a substrate and a liquid portion arranged in a folded configuration and stacked one on top of the other to provide a stack of wet-wipes or interfolded in a configuration suitable for pop-up dispensing. Such folded configurations are well known to those skilled in the art and include c-folded, z-folded, quarter-folded configurations and the like. The stack of folded wet-wipes can be placed in the interior of a container, such as a plastic tub, to provide a package of wet-wipes. Alternatively, the wet-wipes may include a continuous strip of substrate material which has perforations between each wipe and which can be arranged in a stack or wound into a roll for dispensing.

The substrates of use in the present invention are typically non-woven materials produced from cellulosic pulp and/or man-made fibers. The substrates are selected to perform the desired wiping properties for the particular end use application and are to be considered disposable in nature. The substrate is often a combination of more than one type of fiber, such as polypropylene, polyester, viscose, cotton, cellulose, derivatized cellulose, other synthetic fibers, or mixtures thereof; notwithstanding, the substrate could be a single fiber type. The fibers are formed into non-woven, woven or knit fabric wipes by various technologies, such as spun bonding, spun lacing, card and bonding and the like. Many of these substrates utilize thermal bonding to provide the required mechanical resistance between the fibers used to form the substrate.

The production of polymer fibers for nonwoven materials usually involves the use of a mix of at least one polymer with nominal amounts of additives, such as stabilizers, pigments, antacids and the like. The mix is melt extruded and processed into fibers and fibrous products using conventional commercial processes. Nonwoven fabrics are typically made by making a web, and then thermally bonding the fibers together. For example, staple fibers are converted into nonwoven fabrics using, for example, a carding machine, and the carded fabric is thermally bonded. The thermal bonding can be achieved using various heating techniques, including heating with heated rollers, hot air and heating through the use of ultrasonic welding.

Fibers can also be produced and consolidated into nonwovens in various other manners. For example, the fibers and nonwovens can be made by spunbonded processes. Also, consolidation processes can include needlepunching, through-air thermal bonding, ultrasonic welding and hydroentangling.

The gelling agent, such as calcium chloride, can be incorporated by into the substrate by various means. For example, it can be added into a pulp dispenser as a solid in a manner similar to how superabsorbent powders or sodium bicarbonate odor absorbers are added in the production of thermally bonded webs. The gelling agent can be mixed with pulp which is added to make spunlace webs. The gelling agent can be incorporated into a pulp mash for wet-laid webs. The gelling agent can be coated onto a surface of other fibers which comprise web structures which are then held together by either thermal or mechanical means.

The gelling agent is either bonded to the substrate through thermal bonding where fibers of the substrate a melted or partially melted to form the final substrate and where the gelling agent becomes adhered or entrapped to the substrate. An effective amount of the gelling agent is to be available to effectively thicken or gel the continuous phase while the continuous phase is in contact with the substrate. An advantage of this process over previous processes is that the addition, adhesion or entrapment of the gelling agent is accomplished using processes already being employed in the production of substrates and wet-wipes.

The liquid portion is added to the substrate to produce the wet-wipe composition. The liquid portion can be any solution which can be absorbed into the substrate and may include any suitable components which provide the desired wiping properties. For example, the components may include water, emollients, surfactants, fragrances, preservatives, chelating agents, pH buffers or combinations thereof as are well known to those skilled in the art.

The liquid portion of the wet-wipe can be an oil-in-water emulsion or a water-in-oil emulsion or a solution or a suspension or slurry that not only cleanse the body but also soothes and heals the body, especially in babies.

Other ingredients that optionally can be included in the liquid portion of the wet-wipe are stabilizers, water thickeners (such as cellulose ethers) oil phase thickeners and stabilizers, suspending agents, colors, and other benefiting agents. Examples of benefiting agents include oil and fat and their derivatives, conditioning agents, soothing agents, healing agents, insect repellent agents, deodorizing agents, anti-biotics, lubricants, luminance, vitamins, moisturizers, softening agents, cleansing agents, antistatic agents, static agents, and mixtures thereof.

The present wet-wipe composition employs a binary system to control the fluid migration of the liquid portion of wet-wipes when the wet-wipes are arranged in a stack or wound into a roll and stored in a container until use by the consumer.

The binary system comprises (A) thickening or gelling polymer in solution, dispersion or emulsion and (B) an effective amount of thickening or gelling agent. Depending upon the particular thickening or gelling polymer, the thickening or gelling agent may be, for example, a salt solution or pH adjusting solution (as would be required in the case of polymers of acrylic acid) so that upon combination within the wet-wipe a results in thickened/gelled continuous phase.

The list of suitable thickening or gelling polymers is broad and spans both natural and synthetic polymers. The thickening or gelling polymer use level is preferably to be between about 0.05 to about 5 wt % in the liquid portion comprising an aqueous phase, preferably between about 0.10 to about 2 wt %.

Below are several examples of binary pairs of thickening or gelling polymers along with the thickening or gelling agents suitable for the task of thickening/gelling the continuous phase while in contact with the substrate of the wet-wipe for the purpose of controlling fluid migration within the wet-wipe during storage. In these pairs, the thickening or gelling polymer is listed first and the thickening or gelling agent is listed second. The binary pairs may be selected from the group consisting of: sodium alginate and Ca²⁺ containing salts; carboxymethylcellulose and Al³⁺ containing salts; guar gum/guar gum derivatives (such as HP-guar (hydroxypropyl guar), HE-guar (hydroxyethyl guar), CM-guar (carboxymethyl guar), M-guar (methylguar), HPM-guar (hydroxypropylmethyl guar), cationic guar, cationic HM guar (cationic hydrophobically modified guar), anionic HM guar (anionic hydrophobically modified guar), and HM guar (hydrophobically modified guar)) and borax; pectin gum and Ca²⁺ containing salts; carrageenan gum and K⁺ containing salts; cross linked polyacrylic acid and neutralizing base solution; xanthan gum, guar gum, and gellan gum and divalent cations; ionic polymer or surfactant having a charge and polymer or surfactant with an opposite charge, and any combination of binary pairs.

A method for practicing the invention comprises combining the thickening or gelling agent of the binary system within the substrate matrix in a solid form, for example in the sodium alginate and Ca²⁺ containing salts binary system, the Ca²⁺ containing salts may be incorporated into the substrate matrix prior to the addition of the sodium alginate in solution form. In this method, a solution of the Ca²⁺ containing salts may be applied to the substrate through various means, such as by spraying, printing or dip coating and the substrate may be subsequently dried. The liquid portion of the continuous phase containing a desired amount of the sodium alginate in solution form, may then be applied to the dried substrate containing the Ca²⁺ salts thereby permitting the liquid portion of the continuous phase to thicken or gel substrate as it effectively penetrates the body of the substrate.

The means to apply the thickening or gelling polymer in solution, dispersion or emulsion with the liquid portion may be applied to the substrate by any means known in the art, including, dip tanks, sprays, transfer rollers and the like.

The wet-wipes of the present invention can have utility as personal care wipes or as household wipes. The personal care wipes can be divided into different categories such as baby wipes, cosmetic/facial wipes, wet toilet tissue, adult wipes, intimate feminine care, personal cleansing, nail polish removers and hair remover pull strips. The household wipes include categories such as all-purpose applications, furniture cleaning, glass cleaning, etc.

The following example serves to illustrate the invention, parts and percentages being by weight unless otherwise indicated.

### EXAMPLES

### Example 1

The airlaid web of the present invention is prepared by disintegrating or defiberizing a pulp sheet or sheets of cotton linters or other short fibers to produce opened short fibers. Although sheeted pulp sheets are preferred, bulk pulp can be substituted in whole or in part by processing through standard textile fiber opening and dosing systems and air conveying to the forming heads. The opened fibers are then air conveyed to forming heads on the airlaid web forming machine. An interfiber binder, such as a synthetic thermoplastic fiber is also introduced, preferably at the forming head, through a fiber “dosing” system. Preferably, the binder is a thermoplastic fiber. The preferred thermoplastic fiber is Celbond Type 255 Bico fiber from Hoechst Celanese. The Bico fiber has a polyester core (approximately 50% of content of fiber) and an activated co-polyolefin sheath (50% of fiber), wherein “activated” implies that the co-polyolefin sheath has been chemically modified to promote adhesion of dissimilar materials. The Bico fiber has a melting point of 128° C., displays a shrinkage of 3% at 110° C., and has a 3 denier. The Bico fibers have approximately a 5 mm cut length, and are low crimp fibers. Although 5 mm is a preferred thermoplastic fiber length, the fibers may be of any suitable length, generally between about 0.5 and 12 mm, including mixtures of fibers of differing lengths. The purpose of the thermoplastic fiber is to bind the short fibers together upon softening or partial melting of the thermoplastic fiber. After the short fibers are bound together by the softened or partially melted thermoplastic fibers, the web is cooled and the thermoplastic fibers harden, permanently binding the short fibers together.

Any thermoplastic fiber may be used provided that the softening or partial melting temperature is sufficiently low that the cotton or other short fibers are not damaged. Further, the thermoplastic fiber should solidify readily upon minimal cooling. Examples of other suitable thermoplastic fibers include polyolefins such as polyethylene and polypropylene, and polyesters, polyamides, nylons and acrylics.

Although the fiber form is preferred, the thermoplastic material may alternatively be provided in the form of granules, pellets, powder, flakes, chips or any other physical form which allows sufficient intimate contact with the short fibers to permit bonding of the short fibers upon application of heat to the fiber/thermoplastic mixture, and to bond or entrap the calcium chloride or its hydrates into the web.

The fibers of the airlaid web may alternatively be bound by hydroentanglement (or spunlacing) by directing a very fine, high pressure water jet towards the airlaid web to cause an physical entanglement of the fibers, supported by, for example, a 60×40 mesh support wire. Current hydroentanglement processes enable one to achieve a maximum waterjet pressure of 300 bar (4400 psi), a basis weight range of 20-400 gsm, maximum production speed of 300 m/min, a bonding energy of 0.30 KWHlkg, 2% fiber loss and excellent reliability.

The web is prepared by disintegrating by hammermill cotton linter cellulose sheet, Grade INR75 (obtained from Buckeye Cellulose Corporation, Memphis, Tenn.), and air conveying the resultant fibers to forming heads. Hoechst Celanese's Celbond Type 255 Bico fiber (a bicomponent fiber with a polyester core and an activated copolyolefin sheath) is dosed into an air stream and is conveyed to the forming heads and blended with the cotton linter cellulose. The amount of the Bico fiber is 15% of the total weight of the airlaid web. The amount of active calcium chloride is 0.3% of the total weight of the airlaid web.

The resultant airlaid web is lightly calendered to reduce thickness to 3 mm, increase density and slightly increase tensile strength. The web is then passed through thermal bonding ovens. The temperature of the ovens is set to a temperature above the melting point of the Bico fiber, with sufficient dwell time in the ovens to allow completion of bonding to produce a thermally bonded airlaid web, e.g. 130-190° C. and a dwell time of about 30 seconds.

Although the invention has been described with referenced to preferred embodiments, it is to be understood that variations and modifications in form and detail thereof may be made without departing from the spirit and scope of the claimed invention. Such variations and modifications are to be considered within the purview and scope of the claims appended hereto.

What is claimed:

1. A wet-wipe having improved fluid migration properties comprising:
a thickening or gelling polymer;
an effective amount of a thickening or gelling agent which is capable of thickening or gelling the thickening or gelling polymer;
a substrate of woven, nonwoven or knitted fabric; and
a liquid portion comprising an aqueous phase,
wherein the thickening or gelling agent is incorporated into the substrate through means of physical entanglement or thermal bonding.

2. The wet-wipe of claim 1 wherein the thickening or gelling polymer and the thickening or gelling agent comprise a binary pair wherein the binary pair is selected from the group consisting of: sodium alginate and Ca²⁺ containing salts;
carboxymethylcellulose and Al³⁺ containing salts; guar gum/guar gum derivatives (such as HP-guar (hydroxypropyl guar), HE-guar (hydroxyethyl guar), CM-guar (carboxymethyl guar), M-guar (methylguar), HPM-guar (hydroxypropylmethyl guar), cationic guar, cationic HM guar (cationic hydrophobically modified guar), anionic HM guar (anionic hydrophobically modified guar), and HM guar (hydrophobically modified guar)) and borax; pectin gum and Ca²⁺ containing salts;
carrageenan gum and K⁺ containing salts; cross linked polyacrylic acid and neutralizing base solution; xanthan gum, guar gum, and gellan gum and divalent cations; ionic polymer or surfactant having a charge and polymer or surfactant with an opposite charge and any combination of binary pairs.

3. The wet-wipe of claim 1 wherein the thickening or gelling polymer comprises between about 0.05 to about 5 wt % of the liquid portion of the aqueous phase.

4. The wet-wipe of claim 3 wherein the thickening or gelling polymer comprises between about 0.10 to about 2 wt % of the liquid portion of the aqueous phase.

5. The wet-wipe of claim 2 wherein the thickening or gelling polymer comprises sodium alginate and the thickening or gelling agent comprises Ca²⁺ containing salts.

6. The wet-wipe of claim 1, wherein the liquid portion further comprises at least one of the ingredients selected from the group consisting of water, emollients, surfactants, fragrances, preservatives, chelating agents, pH buffers, emulsifiers, water thickeners, oil thickeners, stabilizers, and suspending agents.

7. The wet-wipe of claim 1, wherein the liquid portion further comprises a benefiting agent selected from the group consisting of oil and fat and their derivative, humectants, conditioning agent, soothing agent, healing agent, insect repellent agent, deodorizing agent, biocide, lubricant, softening agent, luminance, vitamins, moisturizer, softening cleaning agent, cleansing agent, fragrance, color, antistatic agent, static agent, and mixtures thereof.

8. The wet-wipe of claim 1, wherein the wet-wipe is a personal care wipe.

9. The wet-wipe of claim 1, wherein the wet-wipe is a household care wipe.

10. A method for producing a wet-wipe comprising the steps of:
a) obtaining a substrate containing an effective amount of a thickening or gelling agent; and
b) applying to the substrate a liquid portion comprising an aqueous phase and a thickening or gelling polymer;
wherein the thickening or gelling agent is incorporated into the substrate through means of physical entanglement or thermal bonding.

11. The method for producing a wet-wipe of claim 10 wherein the liquid portion comprising the aqueous phase and the thickening or gelling polymer is applied to the substrate using a dip tank, sprays or transfer roller.
